(19) United States
(12) Patent Application Publication
(10) Pub. No.: US 2016/0175003 A1
(43) Pub. Date: Jun. 23, 2016

(54) COMPOUND NEEDLE

(71) Applicants: Ronny KAFILUDDI, Loudonville, NY (US); Ersno EROMO, Ghent, NY (US)

(72) Inventors: Ronny KAFILUDDI, Loudonville, NY (US); Ersno EROMO, Ghent, NY (US)

(21) Appl. No.: 14/773,156

(22) PCT Filed: Mar. 5, 2014
(86) PCT No.: PCT/US2014/020706

§ 371 Date: Sep. 4, 2015

Related U.S. Application Data
(60) Provisional application No. 61/772,565, filed on Mar. 5, 2013.
(60) Provisional application No. 61/877,110, filed on Sep. 12, 2013.

(51) Int. Cl. A61B 17/34; A61B 8/08; A61M 25/06

(52) U.S. Cl. CPC A61B 17/3401; A61M 25/06; A61M 25/0643; A61B 8/0841; A61B 17/3403; A61B 2017/3413

(57) ABSTRACT

A compound needle comprising an outer needle shaft having an outer needle tip and an inner needle shaft having an inner needle tip wherein one of the needle tips is sharper than the other needle tip. The outer needle tip can be formed of a flexible material and/or have a flexible/movable connection to the outer shaft, which enables the inner needle tip to be deployed and/or refracted through the outer needle tip.

### CROSS REFERENCE TO RELATED APPLICATIONS

This application is being filed on 5 Mar. 2014, as a PCT International patent application, and claims the benefit of U.S. Provisional Application No. 61/772,565 filed Mar. 5, 2013, and U.S. Provisional Application No. 61/877,110 filed Sep. 12, 2013, the disclosures of which are hereby incorporated by reference in their entireties. To the extent appropriate, a claim of priority is made to each of the above disclosed applications.

### TECHNICAL FIELD

The disclosure relates generally to needles, and more particularly, to a compound needle which can be configured with a sharp tip or a blunt tip.

### BACKGROUND ART

Spinal needles frequently are used to perform diagnostic or therapeutic injections in the spine. Currently needles have either a sharp, pencil point or blunt tip. Sharp tipped needles are the most frequently used. An illustrative procedure which utilizes spinal needles includes a selective nerve root or transforaminal injection in order to deposit medicine in close proximity to the spinal nerves.

A problem with the use of a sharp tipped needle is that it may result in nerve damage when it is placed intra-neurally. In addition to nerve damage, vascular injury also can result from a sharp needle piercing a spinal vessel. Furthermore, particulate matter may be accidentally deposited into a spinal artery (which is in close proximity to the target nerve) during a procedure. Such an accidental deposit may result in obstruction of arterial flow to the spine and subsequently in ischemia of the spinal cord which may result in chronic neuropathic pain and even paralysis.

In order to significantly decrease the incidence of neural or vascular damage, blunt needles were developed. A blunt needle tends to displace the nervous structure or blood vessel, rather than puncturing the structure. A blunt pencil needle tip on a spinal needle may still puncture or damage the nerve or artery, but the chance of such damage is less as compared to a sharp needle tip. It has been clinically shown that the incidence of nerve injuries or injuries to vasculature is significantly less when blunt needles are used compared to the use of sharp needles. However, a spinal needle with a blunt needle tip is unable to penetrate the skin and deeper fascial tissue layers.

As a result, current procedures use a sharp needle followed by a blunt needle. In particular, an angiocatheter (including a sharp needle with a plastic cannula surrounding it) is used initially. The angiocatheter with the sharp needle is able to pierce the skin and deeper tissues easily. Once this angiocatheter is placed in proximity to the target nerve, the sharp needle is withdrawn and the plastic cannula left behind. Subsequently, a blunt spinal needle is advanced thru the plastic cannula towards the target nerve until it is placed in close proximity to the nerve.

However, the blunt spinal needle often still needs to be advanced through fascia due to a limited length of the plastic cannula (e.g., typically approximately 1.5 inches), thereby resulting in a “jerky” motion of the blunt spinal needle as it moves toward the nerve. A fluoroscope and contrast material can be used to verify a position of the blunt needle. In particular, the contrast material will spread along the nerve sleeve indicating correct needle placement. Advancing a blunt needle slightly is not likely to puncture a targeted nerve or close-by blood vessel since the tip of the needle is blunt. However, the “jerky” motion can still result in nerve damage since the nerve may be approximated with a high velocity.

### SUMMARY

In general terms, this disclosure relates to a compound needle including a needle with a sharp needle tip (“sharp needle”) and a needle with a blunt needle tip (“blunt needle”). The sharp needle tip can be formed of a flexible material and/or have a flexible/movable connection to the outer shaft, which enables the blunt needle to be deployed and/or retracted through the sharp needle tip. Various aspects are described in this disclosure, which include, but are not limited to, the following aspects.

One aspect provides a compound needle including one or more features as described herein. Other aspects provide methods, products, systems, and methods of using and generating each, which include a compound needle described herein.

The illustrative aspects are designed to solve one or more of the problems herein described and/or one or more other problems not discussed.

Another aspect is a compound needle comprising: an inner shaft having a first needle tip; and an outer shaft having a second needle tip, wherein one of the first and second needle tips is sharper than the other of the first and second needle tips

### BRIEF DESCRIPTION OF THE DRAWINGS

FIG. 1A is a side view of an example of an inner needle with a distal needle tip that has a blunt end with a rounded edge.

FIG. 1B is a side view of an example of an outer needle with a distal needle tip that has a sharp end.

FIG. 2A is a side view of an example of a compound needle where the inner needle is housed inside the hollow shaft of the outer needle.

FIG. 2B is a front view of the distal needle tip of the outer needle of the compound needle in FIG. 2A.

FIG. 2C is a side view of an example of a compound needle where the inner needle is deployed and extends out from the distal needle tip of the outer needle.

FIG. 2D is a front view of the distal needle tip of both the outer needle and the inner needle of the compound needle in FIG. 2C.

FIG. 3A is a side view of an example of a curved inner needle with a distal needle tip that has a blunt end with a rounded edge.

FIG. 3B is a side view of an example of a curved outer needle with a distal needle tip that has a sharp end.

FIG. 4A is a side view of an example of a curved compound needle where the inner needle is housed inside the hollow shaft of the outer needle.

FIG. 4B is a side view of an example of a curved compound needle where the inner needle is deployed and extends out from the distal needle tip of the outer needle.

FIG. 5A is a side view of an example of an inner needle with helical ridges on the proximal end of the inner needle shaft.

FIG. 5B is a side view of an example of an outer needle with a rotatable member.

FIG. 6 is a schematic view of an example of a compound needle system.

FIG. 7 is a side view of an example of an outer needle with a sharp distal needle tip and a rotatable outer member.

FIG. 8 is a side view of an example of an inner needle with a blunt distal needle tip and a proximal needle hub.

FIG. 9 is a side view of an example of a compound needle where the inner needle of FIG. 8 is inside the outer needle of FIG. 7.

FIG. 10 is a side view of an example of an outer needle with a sharp distal needle tip and a proximal outer member with a ball opening.

FIG. 11 is a side view of an example of an inner needle with a blunt distal needle tip and a proximal needle hub with a spring loaded ball.

FIG. 12 is a side view of an example of a compound needle where the inner needle of FIG. 11 is inside the outer needle of FIG. 10 and the spring loaded ball is aligned and locked in the ball opening.

FIG. 13 is a perspective view of an example of a compound needle with a syringe attached to the proximal end of the proximal needle hub with helical ridges.

FIG. 14 is a perspective view of an example of a compound needle with a syringe attached to the proximal end of the proximal needle hub with a spring loaded ball.

FIG. 15 is a side view of an example of the distal end of a compound needle where the inner needle is housed inside the outer needle.

FIG. 16 is a side view of an example of the distal end of a compound needle where the inner needle is slightly deployed and distal needle tip of the outer needle is in a slightly open configuration.

FIG. 17 is a side view of an example of the distal end of a compound needle where the inner needle is further deployed and the distal needle tip of the outer needle is in a more open configuration.

FIG. 18 is a side view of an example of the distal end of a compound needle where the distal needle tip of the inner needle is fully deployed and the distal needle tip of the outer needle is in a fully open configuration.

FIG. 19 is a side view of an example of the distal end of a compound needle where the inner needle shaft is deployed and further extends from the outer needle.

FIG. 20 is a side view of an example of the distal end of a compound needle where the curved inner needle is housed inside a straight outer needle.

FIG. 21 is a side view of an example of the distal end of a compound needle where the curved inner needle is slightly deployed and distal needle tip of the straight outer needle is in a slightly open configuration.

FIG. 22 is a side view of an example of the distal end of a compound needle where the curved inner needle is further deployed and the distal needle tip of the straight outer needle is in a more open configuration.

FIG. 23 is a side view of an example of the distal end of a compound needle where the distal needle tip of the curved inner needle is fully deployed and the distal needle tip of the straight outer needle is in a fully open configuration.

FIG. 24 is a side view of an example of the distal end of a compound needle where the curved inner needle shaft is deployed and further extended from the straight outer needle.

FIG. 25 is a side view of an example of a compound needle with a plurality of indentations on the outer needle shaft.

FIG. 26A is a cross-sectional view of an example of a pair of indentations on the outer needle shaft.

FIG. 26B is an enlarged view illustrating a portion of FIG. 26A.

FIG. 27 is a top view of an example of one indentation on the outer needle shaft.

FIG. 28 is a cross-sectional side view of an example of an outer needle shaft with a plurality of indentations with varying angles.

FIG. 29 is a close up, cross-sectional side view of three examples of indentations with varying angles to the long axis of the outer needle shaft.

FIG. 30 is a schematic view of an example of ultrasound waves reflecting off the angled indentations of the outer needle shaft.

FIG. 31 is a side view of an example of a compound needle with a plurality of metallic deposits on the outer needle shaft

FIG. 32A is a cross-sectional views of an example of a pair of metallic deposits on the outer needle shaft.

FIG. 32B is an enlarged view illustrating a portion of FIG. 32A.

FIG. 33 is a schematic view of an example of a compound needle system including a nerve stimulation instrument.

FIG. 34 is a side view of an example of a compound needle with an insulating layer on the outer needle.

FIG. 35 is a cross-sectional view of an example of the compound needle with an insulating layer on the outer needle.

FIG. 36 is a flowchart illustrating the use of the compound needle system.

FIG. 37 is a side view of an example of the distal end of the compound needle where the distal needle tip of the outer needle is configured in a single grind or cut point configuration.

FIG. 38 is a side view of an example of the distal end of the compound needle where the distal needle tip of the outer needle is configured in a vet point configuration.

FIG. 39 is a side view of an example of the distal end of the compound needle where the distal needle tip of the outer needle is configured in a lancet point configuration.

FIG. 40 is a side view of an example of the distal end of the compound needle where the distal needle tip of the outer needle is configured in a razor's edge configuration.

FIG. 41 is a side view of an example of the distal end of a straight needle tip of an outer needle with a single grind or cut point configuration and a curved needle tip of an inner needle with a blunt end.

FIG. 42 is a side view of an example of the distal end of a curved needle tip of an outer needle with a single grind or cut point configuration and a curved needle tip of an inner need needle with a blunt end.

FIG. 43 is a side view of an example of the distal end of a straight needle tip of an outer needle with a vet point configuration and a curved needle tip of an inner needle with a blunt end.

FIG. 44 is a side view of an example of the distal end of a curved needle tip of an outer needle with a vet point configuration and a curved needle tip of an inner needle with a blunt end.

FIG. 45 is a side view of an example of the distal end of a straight needle tip of an outer needle with a lancet point configuration and a curved needle tip of an inner needle with a blunt end.

FIG. 46 is a side view of an example of the distal end of a curved needle tip of an outer needle with a lancet point configuration and a curved needle tip of an inner needle with a blunt end.

FIG. 47 is a side view of an example of the distal end of a straight needle tip of an outer needle with a razor's edge configuration and a curved needle tip of an inner needle with a blunt end.

FIG. 48 is a side view of an example of the distal end of a curved needle tip of an outer needle with a razor's edge configuration and a curved needle tip of an inner needle with a blunt end.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

As indicated above, aspects of the disclosure provide a compound needle including a needle with an outer shaft and a sharp needle tip (“sharp needle”) and a needle with a blunt needle tip (“blunt needle”) located inside the shaft of the sharp needle. The blunt needle can serve as a stylet inside the outer sharp needle. The sharp needle tip can be formed of a flexible material and/or have a flexible/movable connection, which enables the blunt needle to be deployed and/or retracted through the sharp needle tip. For example, the blunt needle can be deployed by pushing a hub of the blunt needle so that the blunt needle tip end pushes through the flexible tip of the sharp needle. The deployed blunt needle can be locked into position, e.g., using a spring loaded ball, which extends through an opening in the outer shaft of the sharp needle tip. As used herein, unless otherwise noted, the term “set” means one or more (i.e., at least one) and the phrase “any solution” means any now known or later developed solution.

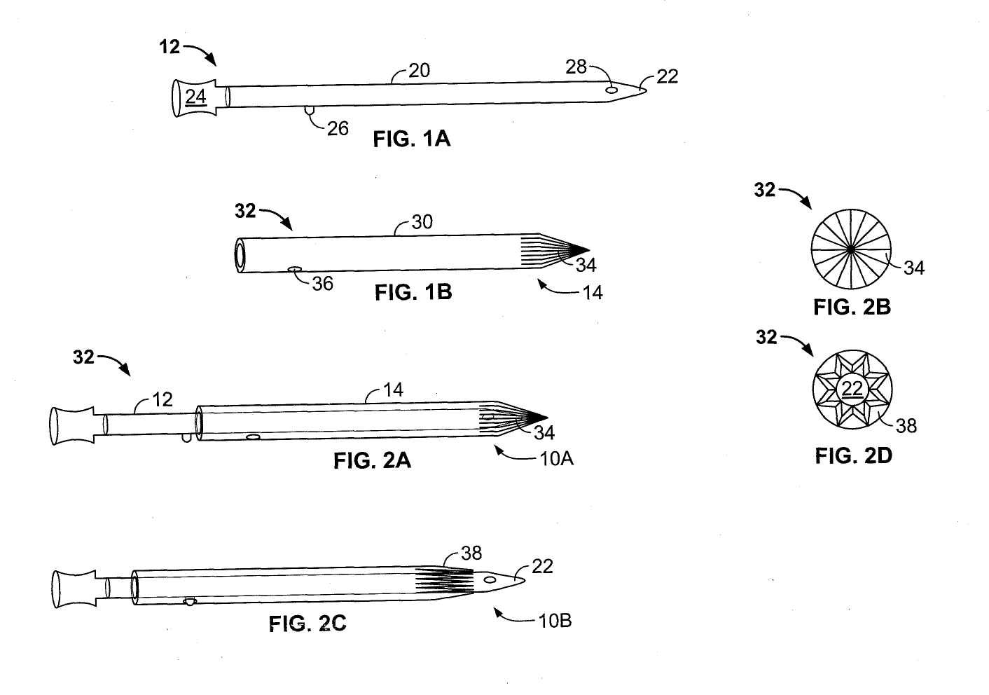

Turning to the drawings, FIGS. 1A and 1B show an illustrative blunt needle 12 and an illustrative sharp needle 14 according to embodiments. The blunt needle 12 can include a shaft 20 having a first end with a blunt needle tip 22 and an opposing end with a hub 24. The sharp needle 14 can include a hollow shaft 30, which terminates at a sharp needle tip 32. In an embodiment, the sharp needle tip 32 includes a plurality of perforating blades 34. The perforating blades 34 can be formed using any solution. For example, a blade 34 can be formed by a cut into the material of the tip 32, formed and attached (e.g., soldered) to the tip 32, and/or the like.

The blunt needle 12 is sized to be located within an interior of the sharp needle 14 to form a compound needle, which can be configured as a blunt needle or a sharp needle. To this extent, FIGS. 2A and 2B show side and front views of an illustrative compound needle in a sharp needle configuration 10A, while FIGS. 2C and 2D show side and front views the illustrative compound needle in a blunt needle configuration 10B according to embodiments. In FIGS. 2A and 2B, the blunt needle 12 is located entirely within the sharp needle 14, and the sharp needle tip 32 forms a closed, pencil like sharpened point on one end of the compound needle. In this configuration, the compound needle can be used to readily pierce tissue using the sharp needle tip 32.

As described herein, the sharp needle tip 32 is configured to allow the blunt needle tip 22 to be deployed there through. As illustrated in FIGS. 2C and 2D, in order to allow the blunt needle tip 22 to deploy, the sharp needle tip 32 can be formed of a plurality of flexible members 38, which can separate as the blunt needle tip 22 is pushed against an interior of the sharp needle tip 32. Furthermore, the flexible members 38 can be configured to return to their original positions when the blunt needle tip 22 is retracted. For example, each flexible member 38 can comprise a corresponding perforating blade 34, which is attached to the sharp needle shaft 30 using a flexible material and/or a flexible connection. The flexible material/connection can comprise any type of material/connector having a sufficient stiffness to enable the sharp needle tip 32 to be utilized to readily pierce tissue, but a sufficient flexibility to allow the blunt needle tip 22 to be deployed through the sharp needle tip 32. In an embodiment, each needle 12, 14 is formed of a material, such as stainless steel or another alloy (e.g., tin), having sufficient strength to resist breaking off while in use (e.g., in tissue).

In an embodiment, the blunt needle 12 can be configured to be locked into a position in the deployed and/or non-deployed positions. To this extent, the blunt needle shaft 20 is shown including a spring loaded ball 26 (FIG. 1A), while the sharp needle shaft 30 is shown including an opening 36 (FIG. 1B). As shown in FIGS. 2A and 2C, when the blunt needle tip 22 is deployed through the sharp needle tip 32, the spring loaded ball 26 can align with the opening 36, thereby locking the blunt needle tip 22 in place. It is understood that the blunt needle 12 and the hollow interior of the sharp needle shaft 30 can be configured to ensure that the spring loaded ball 26 and the opening 36 will align (e.g., by having complementary shapes that only allow the blunt needle 12 to be inserted such that the alignment will occur). Furthermore, it is understood that while only a single opening 36 is shown, the sharp needle shaft 30 can include a plurality of openings, which can correspond to a set of is non-deployed positions for the blunt needle 12 and a set of deployed positions for the blunt needle 12.

In an embodiment, the compound needle can be used to perform a treatment procedure on a target nerve. In an illustrative application, the blunt needle 12 can be used to inject a local anesthetic to anesthetize the target nerve. In another illustrative application, the blunt needle 12 can be used to ablate the target nerve. In either case, the shaft 20 of the blunt needle 12 can be hollow to enable delivery of a treatment to the target nerve through a port 28 (FIG. 1A) located adjacent to the blunt needle tip 22. For example, the local anesthetic can be injected through the hub 24 of the blunt needle 12 and exit the port 28 in a location adjacent to the target nerve. Similarly, a heating probe of a radiofrequency generator can be placed into the blunt needle 12 though the hub 24 and located adjacent to the port 28 to ablate the target nerve. Regardless, the blunt needle 12 and the sharp needle 14 can be configured to enable a sufficient length of the blunt needle 12 to be deployed beyond the sharp needle tip 32 so that the port 28 is also beyond the sharp needle tip 32.

A pencil tipped needle, such as that shown in FIGS. 1A-2D, is not readily steerable. In a further embodiment, compound needles having any one of a plurality of needle tip configurations can be provided. For example, a first needle tip configuration can comprise a straight tip needle, such as that shown and described in conjunction with FIGS. 1A-2D. A second needle tip configuration can comprise a curved tip needle, which can enable an operator to better steer the compound needle towards a targeted nerve. In a still further embodiment, the compound needle can include a combination straight/curved tip needle, where one needle tip (sharp or blunt) is straight or curved, while the other needle tip is the other of straight or curved.

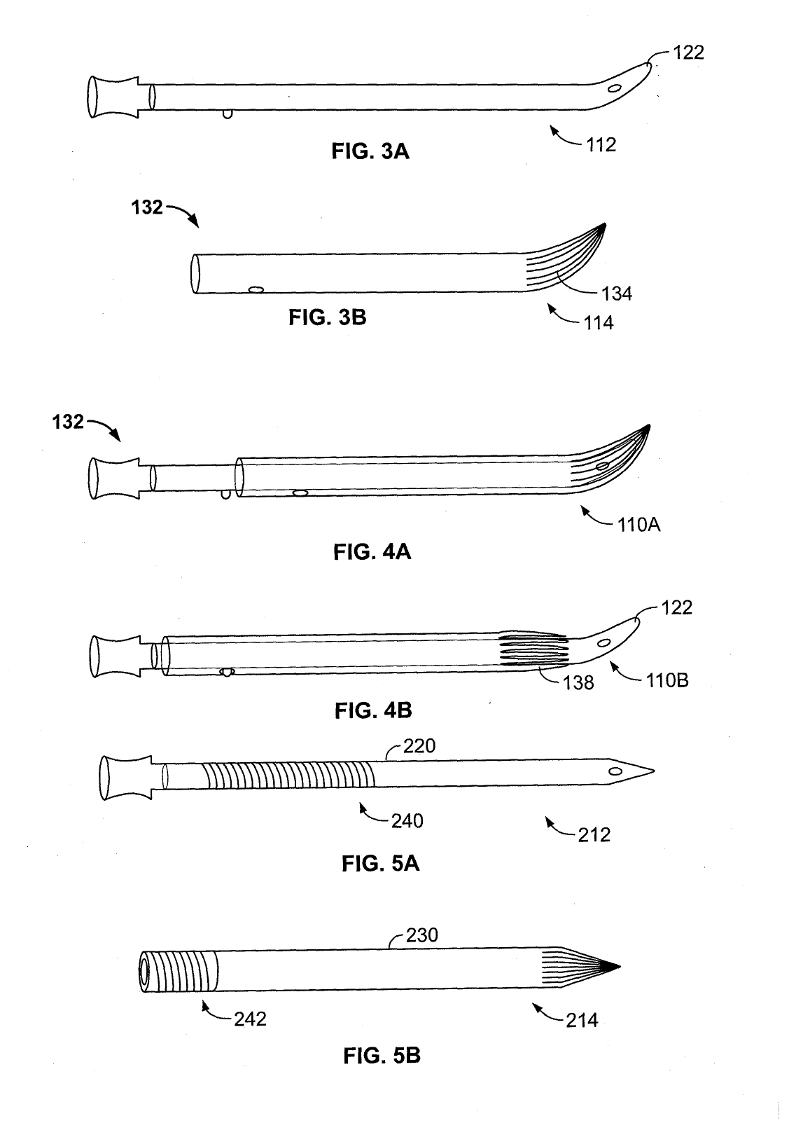

FIGS. 3A and 3B show an illustrative blunt needle 112 and an illustrative sharp needle 114 according to embodiments. Additionally, FIGS. 4A and 4B show an illustrative compound needle formed using the blunt needle 112 and the sharp needle 114 in a sharp needle configuration 110A and a blunt needle configuration 110B according to embodiments. The blunt needle 112 and the sharp needle 114 can include various components and features similar to those described herein in conjunction with FIGS. 1A-2D. As a result, these features and components are not further described for clarity. However, the blunt needle 112 includes a curved blunt needle tip 122 and the sharp needle 114 includes a curved sharp needle tip 132 comprising a plurality of curved perforating blades 134 and a plurality of curved flexible members 138. Use of a curved needle tip 122, 132 can enable an operator to better steer the needle tip 122, 132 towards a targeted nerve. As illustrated, the curved flexible members 138 of the needle tip 122 can be configured to flatten when the blunt needle 112 is deployed there through such that the curved perforating blades 134 are substantially snug to the shaft of the blunt needle 112. In this manner, the curved perforating blades 134 are less likely to damage any tissue surrounding the compound needle. For example, the plurality of flexible members 138 can be configured such that they cannot separate by more than a small amount. In this case, when the shaft of the blunt needle forces the lower flexible members 138 lower, they will pull the upper flexible members 138 down to the shaft of the blunt needle.

As discussed herein, the blunt needle can be deployed through the tip of the sharp needle by pushing on a hub of the blunt needle. However, it is understood that an embodiment of the compound needle can enable finer control of the deployment of the blunt needle and/or continual locking of the blunt needle in a position. For example, FIGS. 5A and 5B show an illustrative blunt needle 212 and an illustrative sharp needle 214 according to embodiments. As previously described, the blunt needle 212 can be configured to be located within a hollow shaft 230 of the sharp needle 214. However, in this case, at least a portion of the outer surface of the shaft 220 of the blunt needle 212 is threaded 240. Additionally, the hollow shaft 230 of the sharp needle 214 can include a rotatable member 242 attached to a distal end thereof. The rotatable member 242 can be attached in a manner that enables an operator to rotate the rotatable member 242 without rotating the shaft 230. The rotatable member 242 can have an interior surface with threading complementary to the threaded portion 240 of the exterior of the blunt needle shaft 220. In this case, an operator (human or computer) can rotate the rotatable member 242 to deploy to and/or retract the blunt needle 212 as described herein. Furthermore, the complementary threading can secure the blunt needle 212 in any of various positions. It is understood that the threaded member and threading described herein is only illustrative of various configurations, which can be utilized to enable finer, continual adjustment of the location of the blunt needle 212 with respect to the sharp needle 214.

Aspects of the needle described herein can enable various procedures, which currently use two different needles and a plastic cannula, to be performed using a single needle, thereby saving time (e.g., one needle entry), expense (e.g., no need for an angiocatheter), and/or the like. A needle described herein can be manufactured in any of various sizes and configurations. For example, a length of the outer needle can vary between approximately 3.5 and approximately 6 inches, and an inner diameter of the inner needle can vary between approximately 18 gauge and approximately 25 gauge (e.g., 18G, 200, 22G, 25G, and/or the like). Regardless, prior to distribution, a needle described herein can be sterilized using any solution and packaged in a sterilized pouch.

A needle described herein can be used in various environments where safe access to a nerve, such as a spinal nerve, a peripheral nerve (e.g., the nerves of the brachial plexus, sciatic nerve, pudental, etc.), and/or the like, is needed. Such environments can include clinical settings such as a medical office, an ambulatory surgery center, a hospital, and/or the like, in which a spinal injection may be performed for diagnostic and/or therapeutic purposes. Specialists who may use the needle include: orthopedic surgeons, neurosurgeons, radiologists, physical medicine and rehabilitation specialists, pain management physicians, and/or the like.

During use of the compound needle, the sharp needle can be used to readily pierce tissue and can be located in close proximity to a targeted nerve/blood vessel using, for example, a fluoroscope, ultrasound, and/or the like. Once the sharp needle is sufficiently close to the targeted nerve/blood vessel but sufficiently far away so as not to puncture the targeted nerve/blood vessel, the blunt needle can be deployed, e.g., by pushing a hub of the blunt needle to extend the blunt needle tip thru the flexible tip of the sharp needle. Once deployed, the blunt needle can be locked into position and steered/advanced, e.g., fluoroscopically with the use of contrast, to a location adjacent to the targeted nerve/blood vessel. Since there usually is no fascial layer around a nerve, the blunt needle can be placed adjacent to the target nerve without a jerky motion, which could damage the target nerve.

In an embodiment, the compound needle can be used to anesthetize or ablate a nerve, such as a peripheral nerve. To this extent, the compound needle can be included as a component of a system for anesthetizing and/or ablating a nerve. For example, using such a system, a local anesthetic can be injected through the blunt needle to anesthetize the nerve, a heating probe from a radiofrequency generator can be placed within the shaft of the blunt needle to ablate the nerve, and/or the like.

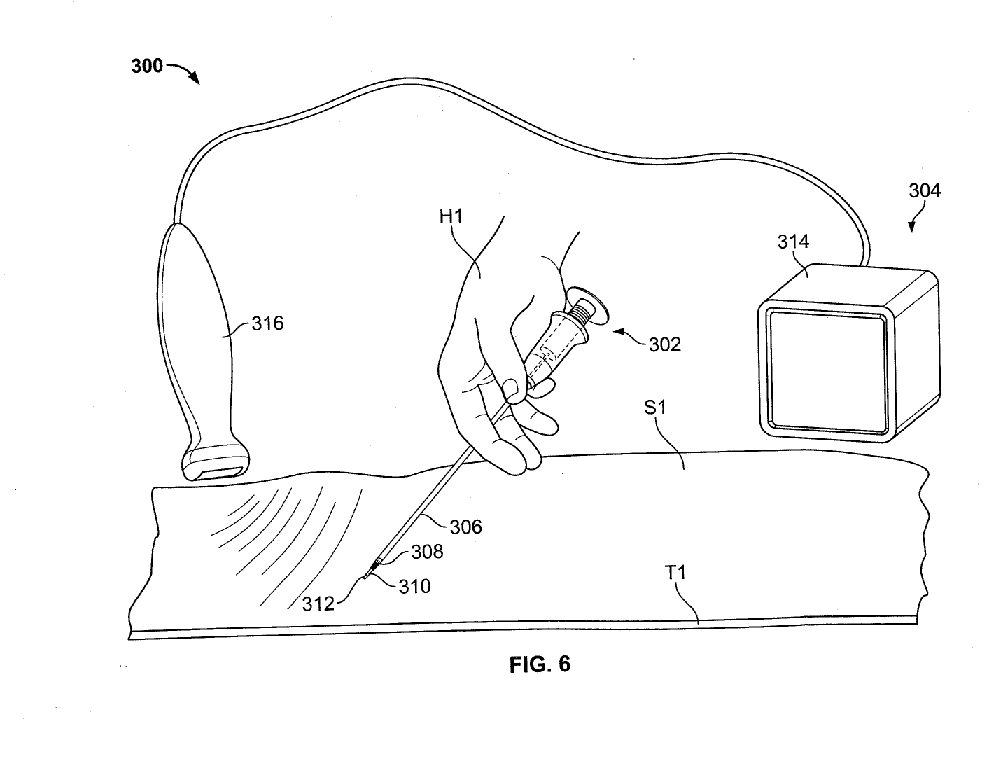

FIG. 6 is a schematic view of an example of a compound needle system 300. In this exemplary embodiment, the compound needle system 300 includes a compound needle 302 and a visual guidance instrument 304. Also illustrated in FIG. 6 is a healthcare provider H1, skin S1, and a target T1. In this embodiment, the compound needle 302 is used in combination with the visual guidance instrument 304 to treat or locate the target T1. Examples of target T1 are a spinal or peripheral nerve or artery. The compound needle and its components will be described in greater detail below.

The compound needle 302 includes an outer needle 306 and an inner needle 310. The outer needle 306 has a distal needle tip 308. The inner needle 310 has a distal needle tip 312. The inner needle 310 is sized to run within the interior of the outer needle 306. Thus, the inner needle 310 is of a longer length than the outer needle 306 in some embodiments. Additionally, in some embodiments the inner needle 310 has an outer diameter less than an outer diameter of the outer needle 306 to permit the inner needle 310 to fit inside of the outer needle 306. Examples of the compound needle 302 are illustrated and described in more detail herein.

In the illustrated embodiment, the visual guidance instrument 304 has an external monitor device 314 and a handheld probe 316. The external monitor device 314 is connected by wire to the handheld probe 316.

In some embodiments, the healthcare provider H1 utilizes the compound needle 302 in combination with the visual guidance instrument 304. In one example, the visual guidance instrument 304 is an ultrasound device in which the handheld probe 316 emits into the skin S1 ultrasonic waves that reflect off the compound needle 302, thereby creating an ultrasound generated image in the external monitor device 314.

In some embodiments, the compound needle 302 allows the visual guidance instrument 304 to more easily detect the compound needle 302 to show the position of the compound needle 302 on the ultrasound image. Furthermore, by utilizing two separate needles with different needle tips, the compound needle 302 allows the healthcare provider H1 to easily pierce the skin and penetrate through layers of fascia without fear of accidentally damaging nerves or vessels.

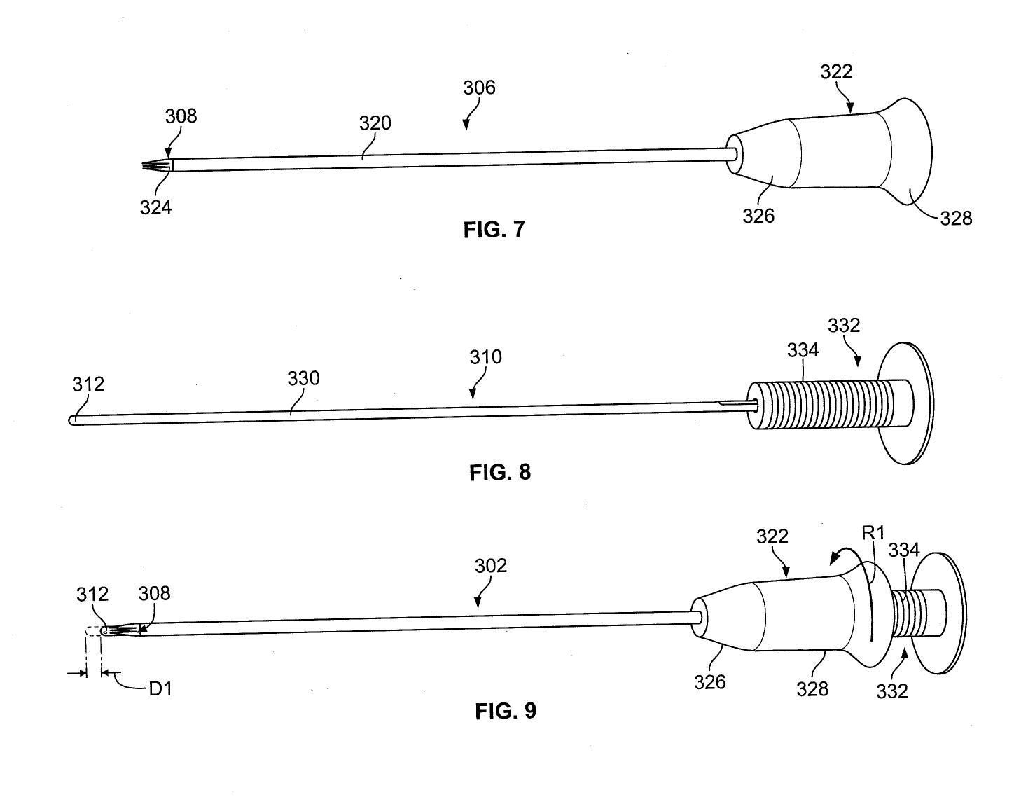

FIGS. 7-9 are side views of an example of a compound needle 302 with a rotatable outer member. FIG. 7 is a side view of the outer needle 306. A variety of needle shapes and configurations are used in different embodiments but in this particular embodiment, the outer needle 306 includes a distal needle tip 308, an outer needle shaft 320, and a rotatable outer member 322. The distal needle tip 308 includes a plurality of perforating blades 324. The rotatable outer member 322 includes a distal end 326 and a proximal end 328.

In this example, the outer needle shaft 320 is made of a material such as metal (e.g., stainless steel) that is shaped and sized to contain an inner needle. In some other embodiments, the outer needle shaft 320 is made of a more pliable material (metal or otherwise) that allows the inner needle shaft to take various shapes and configurations.

On the distal end of the outer needle shaft 320 is the distal needle tip 308. In this example, the distal needle tip 308 is comprised of a plurality of perforating blades 324. The perforating blades 324 are of sufficient stiffness and sharpness to be able to pierce a barrier such as skin or fascia. But the perforating blades 324 must also be flexible enough to allow the inner needle to be deployed or retracted through them, as seen in the Figures below.

On the proximal end of the outer needle 306 is the rotatable outer member 322. In this example, the rotatable outer member 322 is comprised of a proximal end 328 and a distal end 326. The proximal end 328 is rotatable. This is better illustrated in FIG. 9 below. The distal end 326 is not rotatable and is anchored by the proximal end of the outer needle shaft 320.

FIG. 8 is a side view of an example of the inner needle 310. A variety of needle shapes and configurations are used in different embodiments but in this particular embodiment, the inner needle 310 includes a distal needle tip 312, an inner needle shaft 330, and a proximal needle hub 332. The proximal needle hub 332 has a plurality of helical ridges 334.

In this example, the inner needle shaft 330 is made of a material such as metal (e.g., stainless steel) that is shaped and sized to be deployed from within the outer needle shaft 320. In some other embodiments, the inner needle shaft 330 is made of a more pliable material (metal or otherwise) that allows the inner needle shaft to take various shapes and configurations.

On the distal end of the inner needle shaft 330 is the distal needle tip 312. In this example, the distal needle tip 312 is comprised of a blunt end with a rounded edge. On the proximal end of the inner needle, the proximal needle hub 332 has helical ridges 334. The helical ridges 334 complement the screw thread within the rotatable outer member 322 in FIG. 7.

FIG. 9 is a side view of an example of the inner needle 310 of FIG. 8 and the outer needle 306 of FIG. 7 assembled into a compound needle 302. In this embodiment, the distal needle tip 312 of the inner needle is deployed and extends out from the distal needle tip 308 of the outer needle. Because the distal needle tip 312 of the inner needle is deployed, the distal needle tip 308 of the outer needle is in its open configuration. The proximal needle hub 332 is shown to be inside the rotatable outer member 322. The helical ridges 334 are mated to the complementary screw thread within the proximal end 328 of the rotatable outer member 322. The distal needle tip 312 of the inner needle can be further deployed by rotating the proximal end 328 of the rotatable outer member 322, as illustrated by R1. Upon rotation, the distal needle tip 312 of the inner needle extends a predetermined distance D1. In other words, for a given number of rotations in the direction R1, the distal needle tip 312 extends the distance D1 relative to the outer needle shaft 320.

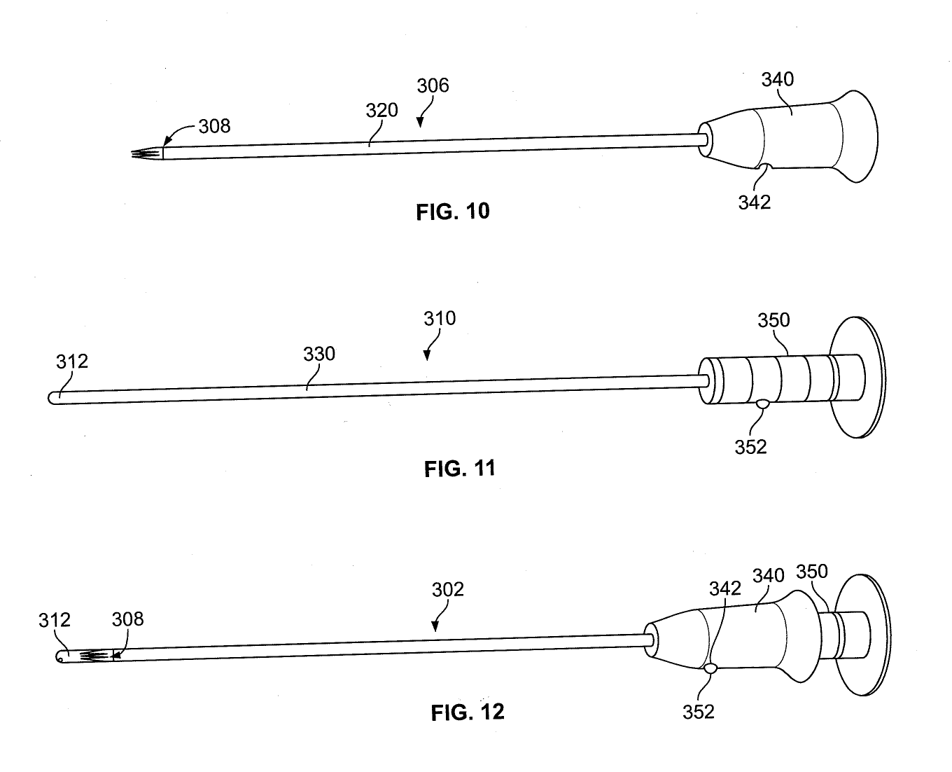

FIGS. 10-12 are side views of an example of the compound needle with a spring loaded ball mechanism. FIG. 10 is a side view of an example of the outer needle 306. A variety of needle shapes and configurations are used in different embodiments but in this particular embodiment, the outer needle 306 includes a distal needle tip 308, an outer needle shaft 320, and a proximal outer member 340 with a ball opening 342.

FIG. 11 is an example of a side view of the inner needle 310. In this particular embodiment, the inner needle 310 includes a distal needle tip 312, an inner needle shaft 330, and a proximal needle hub 350 with a spring loaded ball 352.

In this example, the inner needle shaft 330 is made of a material, such as metal (e.g., stainless steel) that is shaped and sized to be deployed from within the outer needle shaft 320. In some other embodiments, the inner needle shaft 330 is made of a more pliable metal material that allows the inner needle shaft to take various shapes and configurations.

On the distal end of the inner needle shaft 330 is the distal needle tip 312. In this example, the distal needle tip 312 is comprised of a blunt end with a rounded edge. On the proximal end of the inner needle shaft 310, the proximal needle hub 350 has a spring loaded ball 352. The spring loaded ball aligns with the ball opening 342 of the proximal outer member 340 in FIG. 10.

FIG. 12 is a side view of an example of the inner needle 310 of FIG. 11 and the outer needle 306 of FIG. 10 assembled into the compound needle 302. In this embodiment, the distal needle tip 312 of the inner needle is deployed and extends out from the distal needle tip 308 of the outer needle. Because the distal needle tip 312 of the inner needle is deployed, the distal needle tip 308 of the outer needle is in its open configuration. The proximal needle hub 350 is shown to be inside the proximal outer member 340. The spring loaded ball 352 is aligned and locked in the ball opening 342 of the proximal outer member 340. The spring loaded ball 352 positioned within the ball opening 342 locks the inner needle 310 in the desired position.

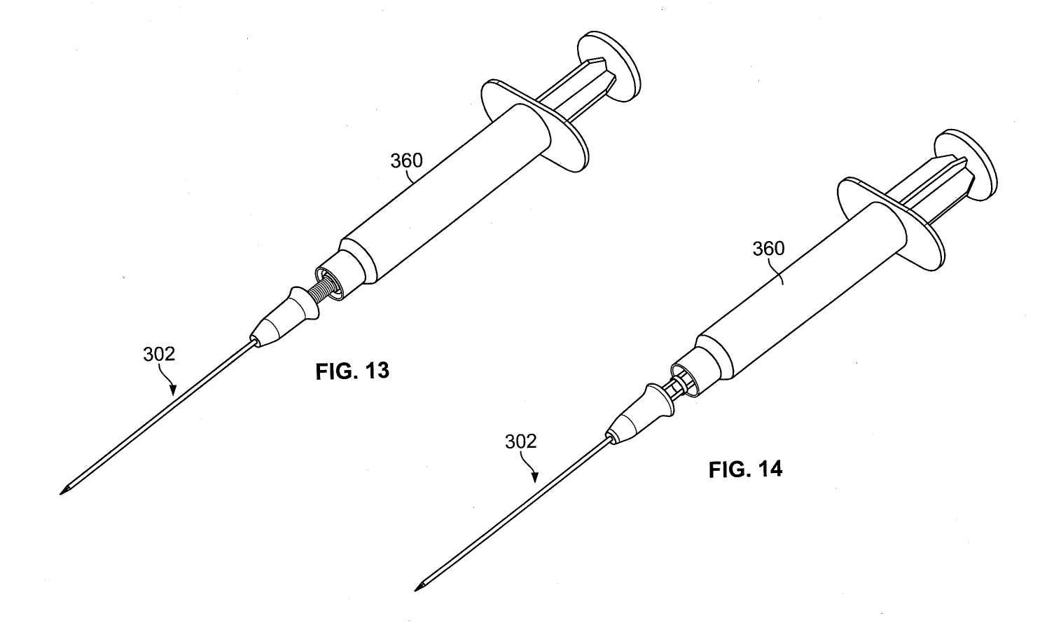

FIGS. 13 and 14 are perspective views of examples of the compound needle 302 attached to a syringe 360. The syringe 360 is fastened to the proximal end of the inner needle and the contents of the syringe are in fluid communication with the hollow interior of the inner needle. The contents of the syringe 360 can therefore be injected into the patient by expressing the contents from the syringe 360, causing the contents to pass through the hub of the inner needle, through the hollow interior of the inner needle, and out from the distal needle tip.

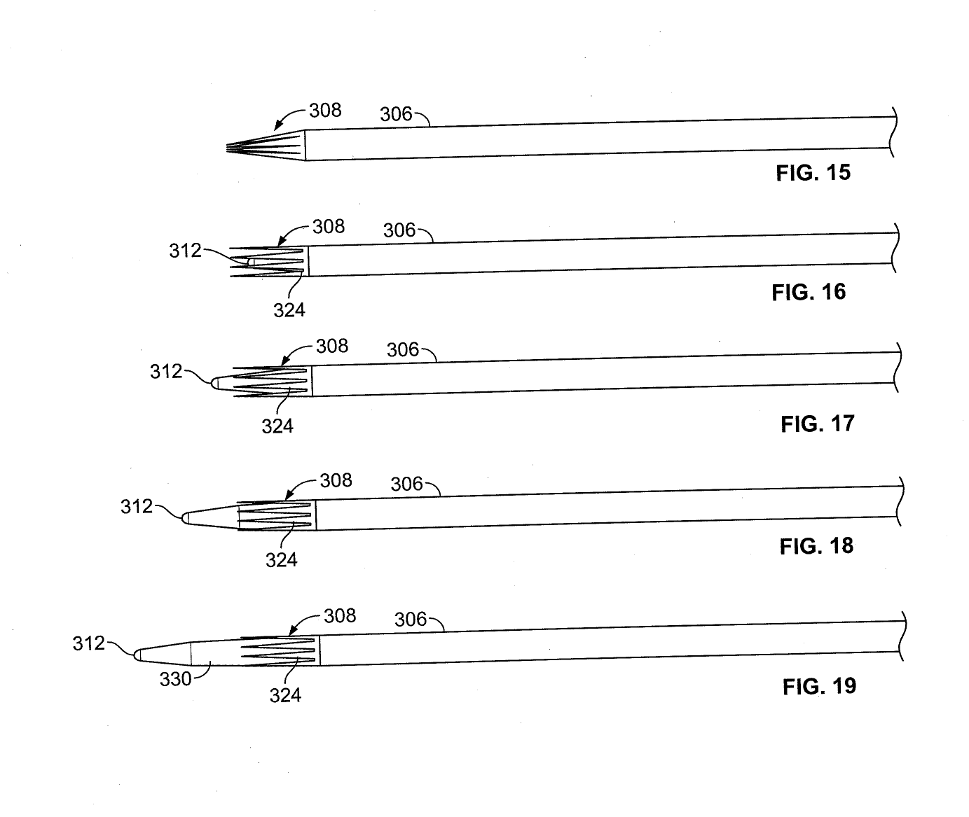

FIGS. 15-19 are side views of examples of the compound needle. In this particular embodiment, both the inner needle 310 and outer needle 306 are configured to be structurally rigid and straight. The inner needles include the distal needle tip 312 and the inner needle shaft 330. Portions of the inner needle 310 are hidden from view in FIGS. 15-19. In FIG. 15, the inner needle 310 is located inside the outer needle 306. The distal needle tip 308 of the outer needle is in a closed configuration.

In FIG. 16, the distal needle tip 312 of the inner needle 310 is slightly deployed. As such, the distal needle tip 308 of the outer needle 306 is in a slightly open configuration and the distal needle tip 312 of the inner needle 310 is visible between the perforating blades 324. In FIG. 17, the distal needle tip 312 of the inner needle 310 is deployed even further. Consequently, the distal needle tip 308 of the outer needle 306 is in a more open configuration. In FIG. 18, the distal needle tip 312 of the inner needle 310 is fully deployed, and the distal needle tip 308 of the outer needle 306 is in a fully open configuration. Lastly, FIG. 19 illustrates the inner needle shaft 330 as also deployed and further extended from the outer needle 306.

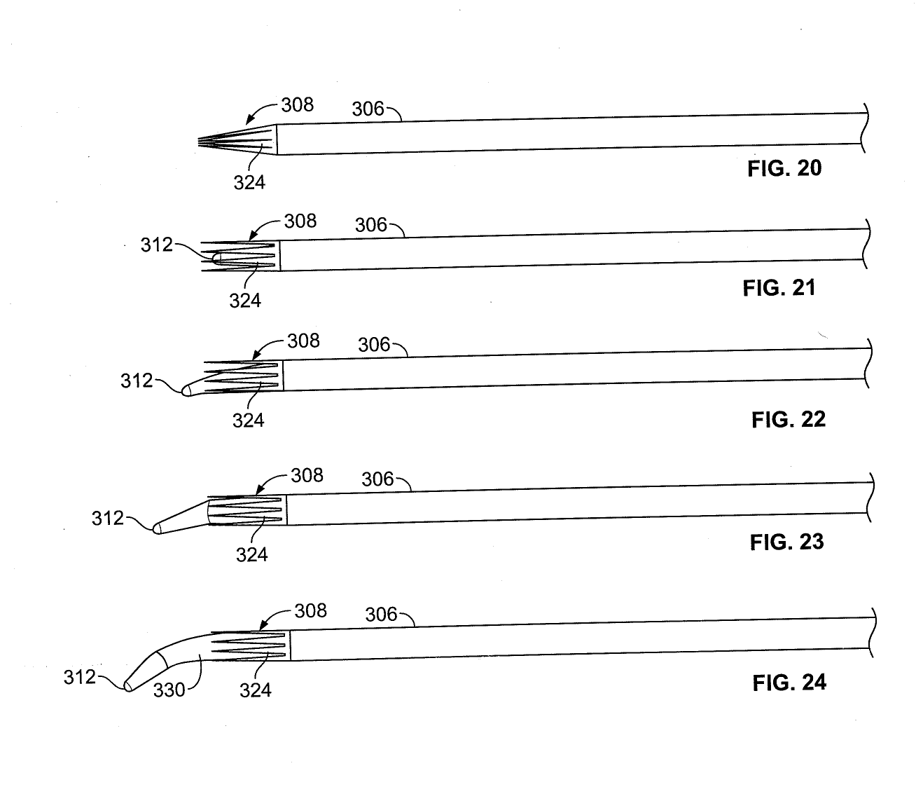

FIGS. 20-24 are side views of examples of the compound needle. In this particular embodiment, the outer needle 306 is configured to be structurally straight but the inner needle 310 is curved when deployed. Portions of the inner needle 310 are hidden from view by the outer needle 306 in FIGS. 20-24. In FIG. 20, the inner needle 310 is located inside the outer needle 306. When the inner needle 310 is located inside the hollow shaft of the outer needle 306, the inner needle 310 can be configured to be straight. In FIG. 21, the distal needle tip 312 of the inner needle is slightly deployed. The distal needle tip 308 of the outer needle is in a slightly open configuration and the distal needle tip 312 of the inner needle is slightly visible between the perforating blades 324. The inner needle 310 still retains a straight configuration. In FIG. 22, the distal needle tip 312 of the inner needle is deployed further. The distal needle tip 308 of the outer needle is in a more open configuration and the distal needle tip 312 of the inner needle is starting to exhibit a curved configuration. In FIG. 23, the distal needle tip 312 of the inner needle is fully deployed, and the distal needle tip 308 of the outer needle is in a fully open configuration. The curved configuration of the distal needle tip 312 of the inner needle is more apparent. Lastly, FIG. 24 illustrates the inner needle shaft 330 as also deployed. A variety of needle shapes and configurations can be used in different embodiments but in the particular embodiments of FIGS. 20-24, the distal needle tip 312 of the inner needle is blunt and with a rounded edge. Conversely, the distal needle tip 308 of the outer needle is sharp with a plurality of perforating blades 324.

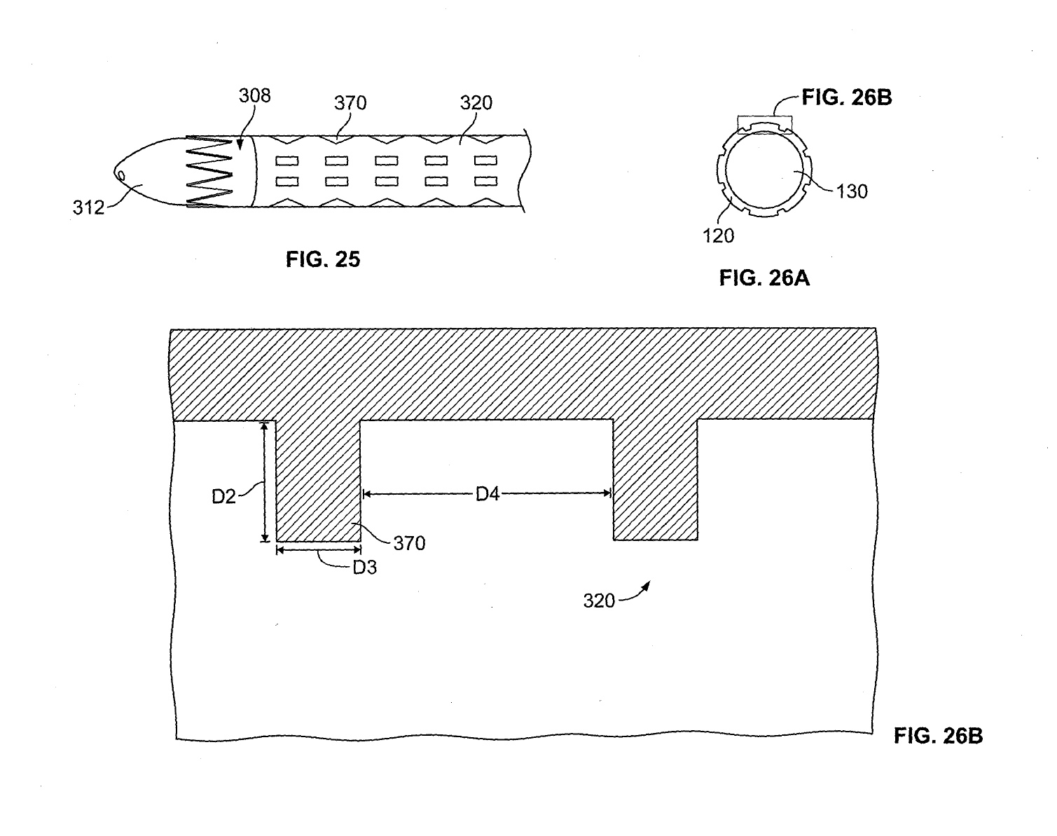

FIG. 25 is a side view of an example of a compound needle with an echogenic shaft. In this embodiment, the distal needle tip 312 of the inner needle is deployed and the distal needle tip 308 of the outer needle is in its open configuration. Indentations 370 are formed on the outer needle shaft 320. In some embodiments the indentations improve the ability of the visual guidance instrument 304 to detect and visually depict the location of the compound needle.

FIGS. 26A and 26B are cross-sectional views of an example of the outer needle shaft 320. The Figures illustrate a pair of indentations 370 on the outer needle shaft 320.

The indentations 370 can be of a variety of different shapes. For example, the indentations can be squared, angled, or curved. In some embodiments the indentations 370 are multi-sided, semi-spherical, semi-elliptical, or have other shapes.

The indentations 370 are illustrated with a depth D2 ranging between 10 micrometers to 100 micrometers. In other embodiments, the depth D2 ranges between 1 micrometer to 1 millimeter. The width D3 of each indentation is at least 10 micrometers. In other embodiments, the width D3 ranges from 1 micrometer to 1 millimeter. The distance D4 is the distance between indentations. In one embodiment, the distance D4 ranges between 10 micrometers and 100 micrometers. In another embodiment, the distance D4 ranges between 1 micrometer and 1 millimeter.

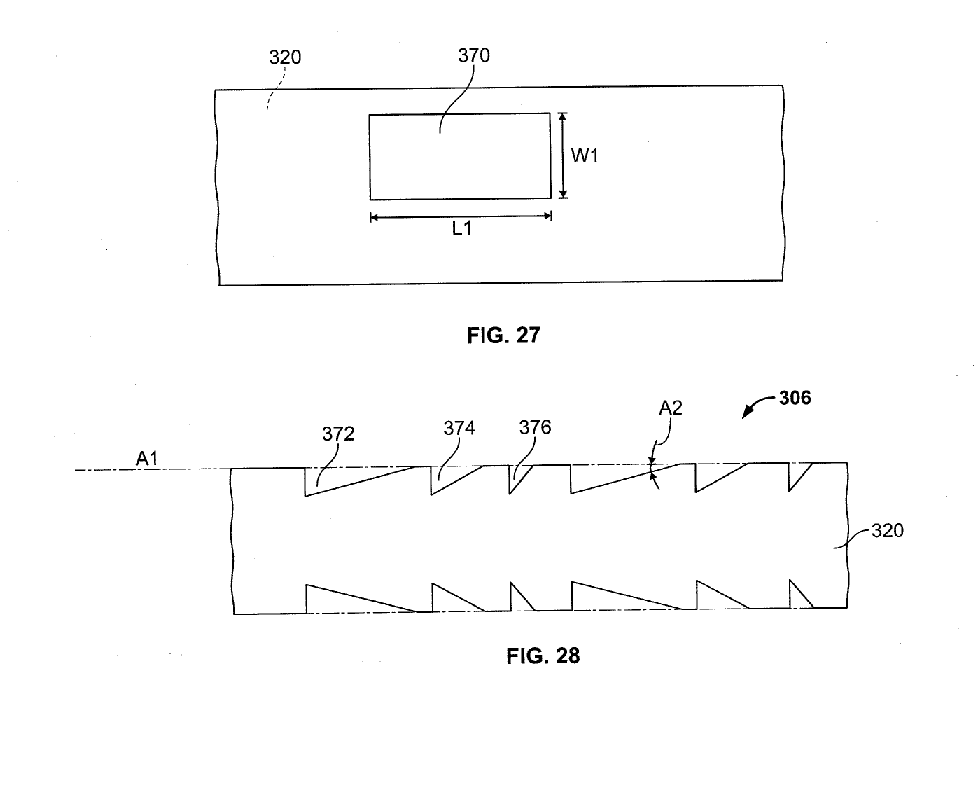

FIG. 27 is a top view of an example of one indentation 370 on the outer needle shaft 320. As illustrated in FIG. 27, the width W1 of the indentation is at least 10 micrometers. In another embodiment, the width W1 ranges between 1 micrometer and 1 millimeter. The length L1 of each indentation is at least 10-100 micrometers. In another embodiment, the length L1 ranges between 1 micrometer and 1 millimeter. The width WI in FIG. 27 corresponds to the D3 in FIG. 26.

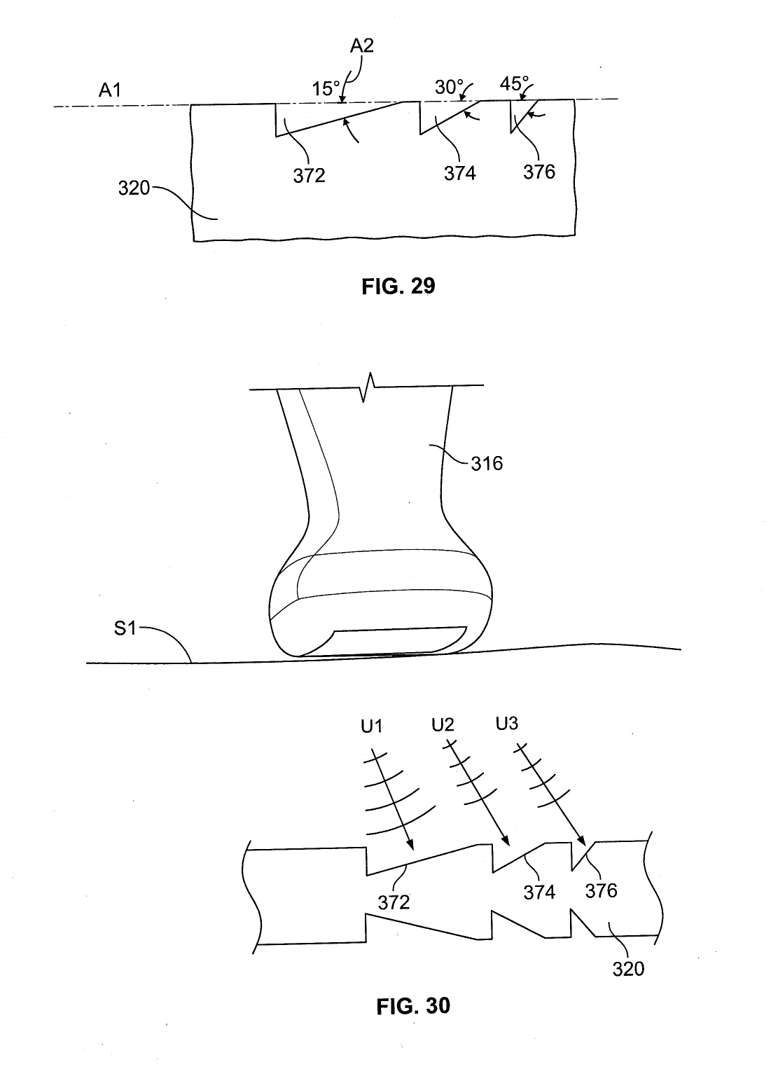

FIG. 28 is a cross-sectional side view of an example of an outer needle 306 with a plurality of indentations at varying angles A2 to the long axis A1 of the outer needle shaft 320. As illustrated in FIG. 28, the indentations are configured be at different angles A2 to the long axis A1 of the outer needle shaft 320. A variety of angles A2 and configurations are used in different embodiments. In this particular embodiment, the first indentation 372 is at a smaller angle to the long axis A1 of the outer needle shaft 320. The second indentation 374 is at a larger angle to the long axis A1. The third indentation 376 is at the largest angle to the long axis A1. FIG. 29 is a close up, cross-sectional, side view of three examples of the indentations on the outer needle shaft 320. As illustrated in the embodiment in FIG. 29, each indentation is at an angle A2 to the long axis A1 of the outer needle shaft 320. The first indentation 372 is at 15 degrees to the long axis A1. The second indentation 374 is at 30 degrees to the long axis A1. The third indentation 376 is at 45 degrees to the long axis A1. In other embodiments, the angle A2 to the long axis A1 ranges from 5 to 90 degrees. In yet other embodiments, the angle A2 to the long axis A1 ranges from 10 to 70 degrees.

FIG. 30 is a schematic view of an example of a compound needle system where three examples of ultrasound waves reflect off the angled indentations of the outer needle shaft 320. Illustrated in FIG. 30 are a handheld probe 316, the first indentation 372, the second indentation 374, the third indentation 376, and the outer needle shaft 320. Also illustrated is the skin S1, a first ultrasound wave U1, a second ultrasound wave U2, a third ultrasound wave U3, and a handheld probe 316. In this embodiment, the handheld probe 316 emits ultrasound wave in various directions depending on the position of the handheld probe. If the handheld probe 316 is situated in a first position, it emits an ultrasound wave U1 that runs perpendicular to the first indentation 372. If the handheld probe is situated in a second position, it emits an ultrasound wave U2 that runs perpendicular to the second indentation 374. If the handheld probe is situated in a third position, it emits an ultrasound wave U3 that runs perpendicular to the third indentation 376.

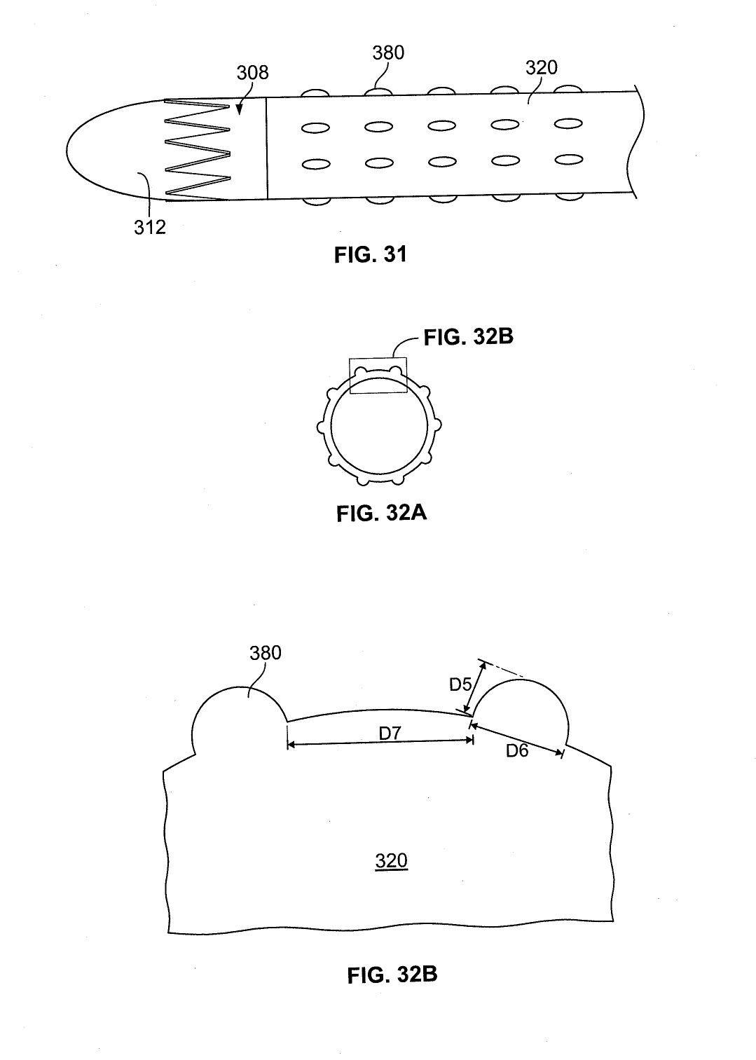

FIG. 31 is also a side view of an example of a compound needle with an echogenic shaft. In this embodiment, the distal needle tip 312 of the inner needle is deployed and the distal needle tip 308 of the outer needle is in its open configuration. Evenly spaced on the outer needle shaft 320 are metallic deposits 380. FIGS. 32A and 32B are cross-sectional views of an example of the outer needle shaft 320. Illustrated are the metallic deposits 380 with a height D5 ranging between 10 to 100 micrometers. In another embodiment, the height D5 ranges between 1 micrometer and 1 millimeter. The width D6 is the distance of the width of one metallic deposition. In one embodiment, D6 is at least 10 micrometers. In another embodiment, the D6 ranges between 1 micrometer and 1 millimeter. The distance D7 is the distance between adjacent deposits. In one embodiment, the distance D7 ranges between 10 micrometers and 100 micrometers. In another embodiment, the distance D7 ranges between 1 micrometer and 1 millimeter.

Both the indentations and the metallic deposits improve the echogenicity of the outer needle shaft 320. For example, when a healthcare provider uses the compound needle with an ultrasound guidance instrument, the metallic deposits or the indentations optimally reflect ultrasound waves emitted from the handheld probe. Thus, the visual guidance instrument is able to generate a clearer image of the compound needle. This ultimately allows for greater precision during medical procedures. Furthermore, the plurality of indentations at varying angles to the long axis A1 allows for optimal reflection of ultrasound waves regardless of the direction in which the ultrasound probe 316 points. It is understood that greatest reflection occurs when the ultrasound waves run perpendicular to the object of reflection (e.g., normal to the surface of the indentation).

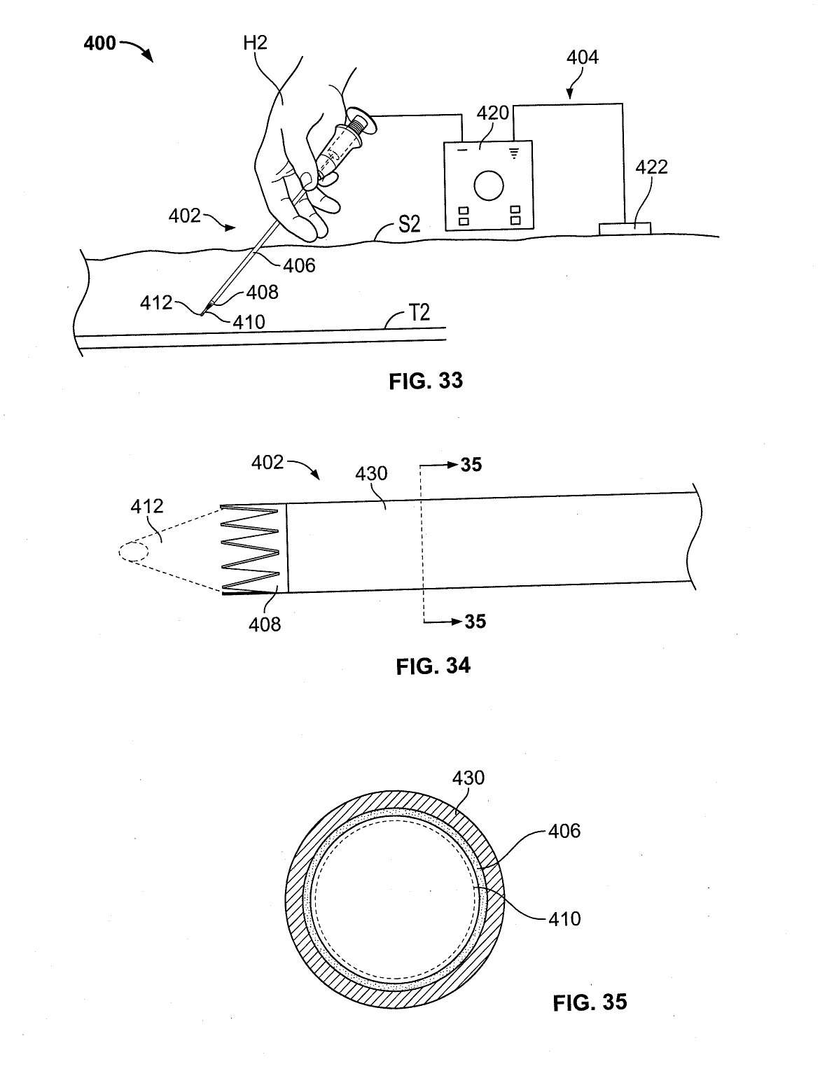

FIG. 33 is a schematic view of an example of a compound needle system 400 including a nerve stimulation instrument 404. The compound needle system 400 provides everything needed to accurately locate or treat target anatomy using electrical stimulation. In this exemplary embodiment, the compound needle system 400 includes a compound needle 402 and a nerve stimulation instrument 404. Also illustrated are a healthcare provider H2, skin S2, and a target T2. In this embodiment, the compound needle 402 is used in combination with the nerve stimulation instrument 404 to treat or locate the target T2. Examples of target T2 is a spinal or peripheral nerve or artery.

The compound needle 402 includes an outer needle 406 and an inner needle 410. The outer needle 406 has a distal needle tip 408. The inner needle 410 has a distal needle tip 412. The inner needle 410 is sized to run along the interior of the outer needle 406. Thus, the inner needle 410 is of a longer length than the outer needle 406. In this embodiment, the nerve stimulation instrument 404 has a stimulation control device 420 and a grounding pad 422. The stimulation control device 420 is connected by wire to the grounding pad 422. The stimulation control device 420 is also in electrical communication with the inner needle 410.

As illustrated, the healthcare provider H2 utilizes the compound needle 402 in combination with the nerve stimulation instrument 404. In this embodiment, the stimulation control device 420 is in electrical connection with the inner needle 410 of the compound needle 402. The stimulation control device 420 generates an electrical signal into the inner needle 410 and consequently into target T2. If the target T2 is a peripheral nerve, a corresponding muscle will be innervated and thus, result in a jerking motion. This effect will assist the healthcare provider H2 in locating the position of the needle 402 with respect to the target T2.

FIG. 34 is a side view of an example of a compound needle 402 with an insulating layer 430 on the outer needle 406. In this embodiment, the distal needle tip 412 of the inner needle is deployed and the distal needle tip 408 of the outer needle is in its open configuration. An insulating layer 430 is evenly placed on the outer needle shaft 406. FIG. 35 is a cross-sectional view of an example of the compound needle with an insulating layer 430 on the outer needle shaft 406.

In this embodiment, the insulating layer 430 on the outer needle shaft 406 is made of a thin plastic film. This insulating plastic film will help concentrate the stimulating electric signal toward the distal needle tip 412 of the inner needle. Thus, this focuses the electric stimulation on the target instead of the surrounding tissue or muscles. The insulating layer can also be made of other material such as silicone rubber, ethylene propylene diene monomer rubber, or other composite insulating materials.

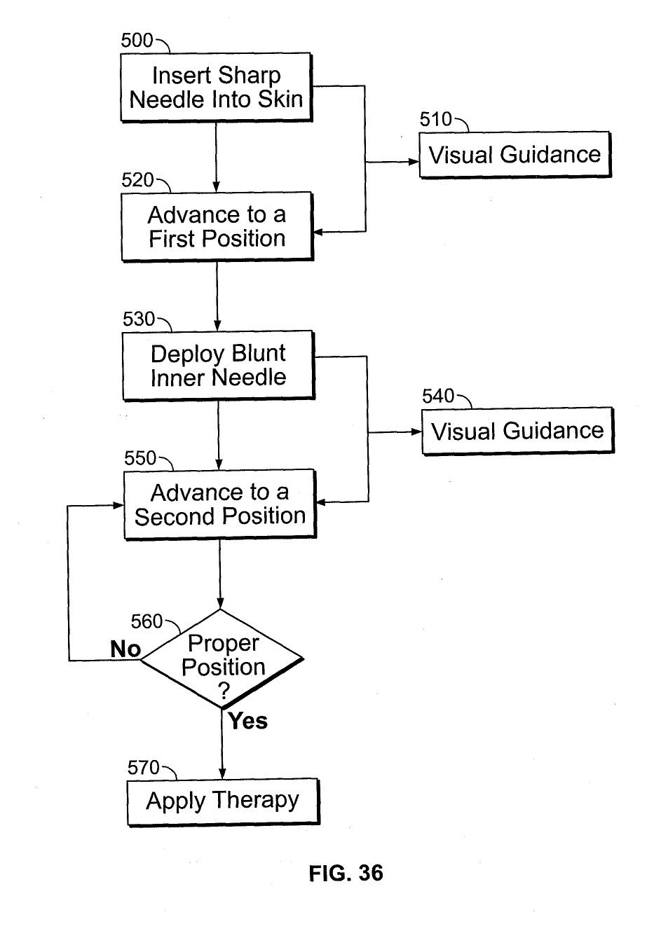

FIG. 36 is a flowchart illustrating the use of the compound needle system. In this embodiment, we have assumed that the compound needle has already been assembled to include, among the other components, the inner needle and the outer needle. Also, we assume that the configuration of the compound needle is such that the inner needle has a distal needle tip with a blunt, rounded end and the outer needle has a distal needle tip that is sharp with a plurality of perforating blades. The configuration of the compound needle could also be arranged in such a manner that the inner needle has a distal needle tip with a sharp end and the outer needle has a distal needle tip with a blunt end, in yet other embodiments.

In FIG. 36, a healthcare provider inserts the skin with the sharp distal needle tip of the compound needle 500. Using the visual guidance instrument 510, the healthcare provider can approximate the position of the compound needle and advance it to a first position 520. Examples of visual guidance instruments include an ultrasound device or a nerve stimulation device. Once at the first position, the healthcare provider can then deploy the inner needle 530. Using the visual guidance instrument, the healthcare provider can approximate the position of the deployed inner needle 540. Once the healthcare provider knows the approximate position of the deployed inner needle, he can then advance the compound needle to a second position 550. In this particular embodiment, the deployed inner needle has a distal needle tip that is blunt and rounded. Thus, the healthcare provider is assured that accidental damage or injury to surrounding nerves and vessels is minimal or unlikely. If the healthcare provider believes that the compound needle is in a proper position 560, he can then apply the appropriate therapy 570. Examples of therapy may include injection of medicine, electrical nerve stimulation, or blunt dissection or ablation of damaged nerves or tissue etc.

If on the other hand, the healthcare provider decides that the compound needle is not in the proper position, he can continue to advance the compound needle to the correct second position using continuous visual guidance 540.

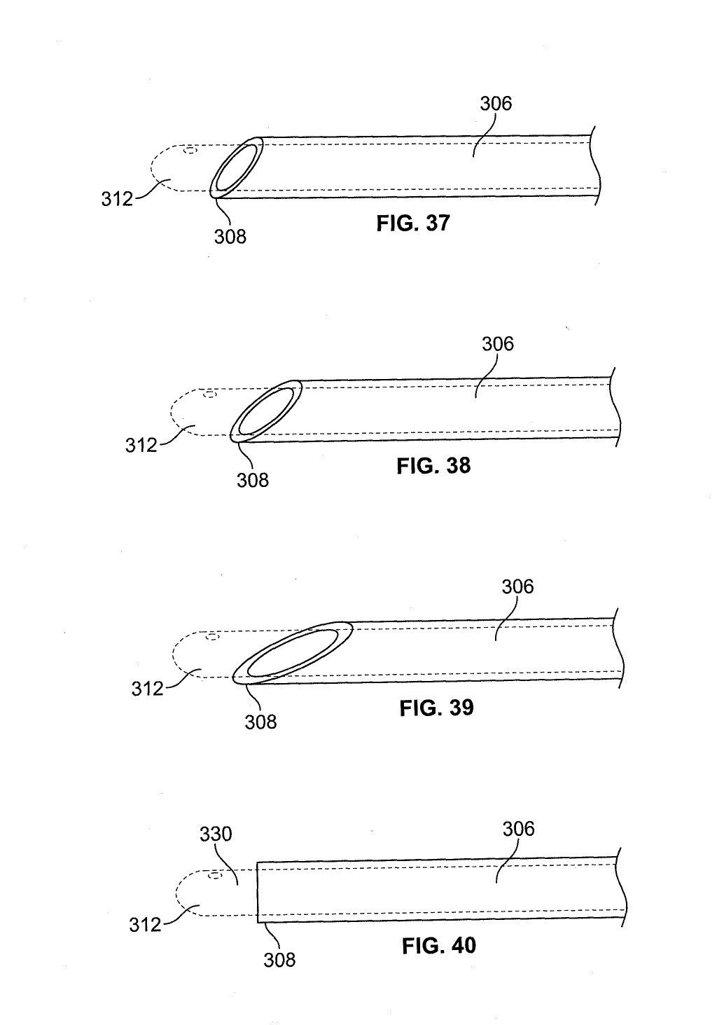

FIGS. 37-40 are side views of examples of the compound needle. In these particular embodiments, both the inner needle 310 and outer needle 306 are configured to have straight needle tips. The inner needles include the straight, distal needle tip 312 and the inner needle shaft 330. Portions of the inner needle 310 and the inner needle shaft 330 are hidden from view in FIGS. 37-40. The outer needle 306 includes the straight, distal needle tip 308 and the outer needle shaft 320.

In FIG. 37, the distal needle tip 308 of the outer needle is in a single grind or cut point configuration. Examples of a single grind or cut point configurations may be a bevel at a 45 degree angle to the shaft. In FIG. 38, the distal needle tip 308 of the outer needle is in a vet point configuration. Examples of a vet point configurations may be a bevel at a 12-15 degree, 18-20 degree or 25-30 degree angle to the shaft and where the points are sharpened on the lumen side of the needle. In FIG. 39, the distal needle tip 308 of the outer needle is in a lancet point configuration. Examples of a lancet point configuration may be a distal needle tip that is sharpened on the lumen fronts with consistent geometry across the tip. Lancet point configuration may be at various angles to the shaft. In FIG. 40, the distal needle tip 308 of the outer needle is in a razor's edge configuration. Examples of a razor's edge configurations may be a distal needle tip 308 that is perpendicular to the shaft and the points are sharpened on the lumen side of the needle.

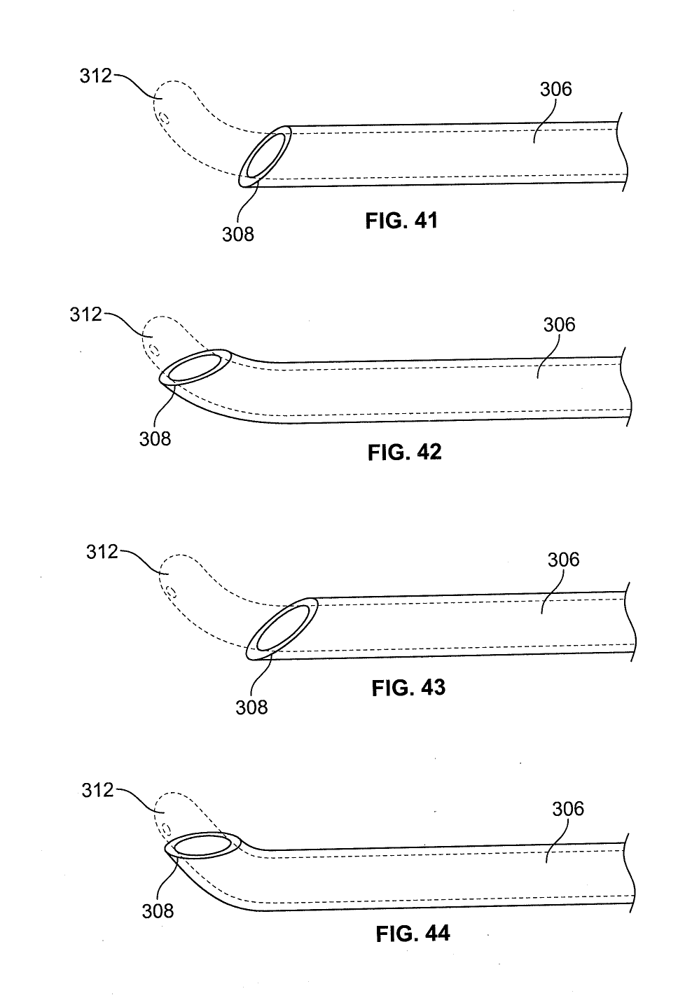

FIGS. 41-48 are side views of examples of the compound needle. In these particular embodiments, the compound needle can include a combination of curved needle tips or a combination of straight and curved needle tips. In one configuration, the needle tip 312 of the inner needle is curved while the needle tip 308 of the outer needle is straight. In this configuration, the needle tip 312 of the inner needle is sufficiently flexible that the needle tip 312 of the inner needle can conform to the shape of the needle tip 308 of the outer needle when the inner needle is disposed inside the hollow core of the outer needle 306. Additionally, the needle tip 312 of the inner needle has shape memory such that when deployed from the needle tip 308 of the outer needle, the needle tip 312 of the inner needle returns to being curved.

Alternatively, both the needle tip 312 of the inner needle and the needle tip 308 of the outer needle may be curved. Though both needle tips are curved, they may or may not have the same degree of curvature. Thus, the needle tip 312 of the inner needle is sufficiently flexible that the needle tip 312 of the inner needle can conform to the shape of the needle tip 308 of the outer needle when the inner needle is disposed inside the hollow core of the outer needle 306. Additionally, the needle tip 312 of the inner needle has shape memory such that when deployed from the needle tip 308 of the outer needle, the needle tip 312 of the inner needle returns to own curved shape.

In FIGS. 41-42, the distal needle tip 308 of the outer needle is in a single grind or cut point configuration. In FIG. 41, the needle tip 312 of the inner needle is curved while the needle tip 308 of the outer needle 306 is straight. In FIG. 42, both the needle tip 312 of the inner needle and needle tip 308 of the outer needle 306 are curved.

In FIGS. 43-44, the needle tip 308 of the outer needle is in a vet point configuration. In FIG. 43, the needle tip 312 of the inner needle is curved while the needle tip 308 of the outer needle 306 is straight. In FIG. 44, both the needle tip 312 of the inner needle and needle tip 308 of the outer needle 306 are curved.

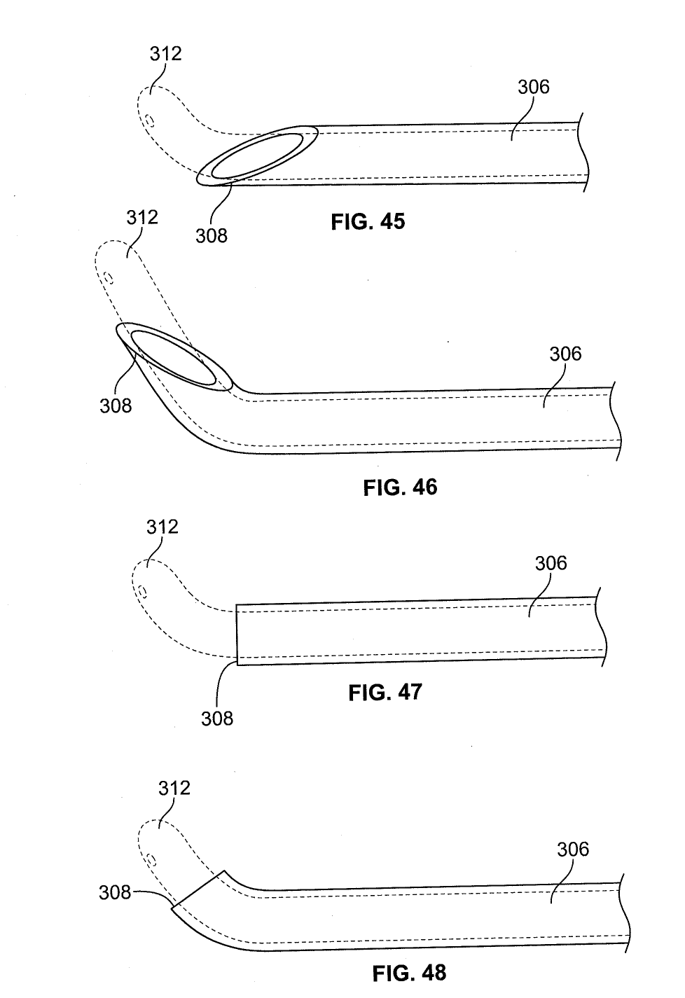

In FIGS. 45-46, the distal needle tip 308 of the outer needle is in a lancet point configuration. In FIG. 45, the needle tip 312 of the inner needle is curved while the needle tip 308 of the outer needle 306 is straight. In FIG. 46, both the needle tip 312 of the inner needle and needle tip 308 of the outer needle 306 are curved.

In FIGS. 47-48, the distal needle tip 308 of the outer needle is in a razor's edge configuration. In FIG. 47, the needle tip 312 of the inner needle is curved while the needle tip 308 of the outer needle 306 is straight. In FIG. 48, both the needle tip 312 of the inner needle and needle tip 308 of the outer needle 306 are curved.

Furthermore, it is understood that the distal needle tip 308 of the outer needle or the distal needle tip 312 of the inner needle can take additional configurations. The various embodiments described above are provided by way of illustration only and should not be construed to limit the shape and configuration of the distal needle tips.

It is also understood that the needle tip can take various blunt configurations. Examples of blunt end needle tips may be a closed blunt end where the blunt distal needle tip is configured to be closed and flat while the shaft retains a lumen space. Another example may be a ball end distal needle tip where the blunt distal needle tip is closed by a spherical ball. Another example may be a bullet point distal needle tip where the blunt distal needle tip is tapered but non-sharp.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

1.-8. (canceled)

9. A compound needle comprising:
an inner shaft having a first needle tip;
an outer shaft having a second needle tip;
wherein one of the first and second needle tips is sharper than the other of the first and second needle tips;
wherein the outer shaft has a hollow core and wherein the inner shaft is disposed within the hollow core; and
wherein the first needle tip of the inner shaft is deployable through the second needle tip of the outer shaft.

10. The compound needle of claim 9, wherein the first needle tip is a blunt tip and the second needle tip is a sharp tip and wherein the sharp tip is sharper than the blunt tip.

11. The compound needle of claim 10, wherein the sharp tip requires less force than the blunt tip to advance through human tissue.

12. The compound needle of claim 10, wherein the sharp tip further comprises perforating members.

13. The compound needle of claim 9, wherein the first needle tip is a sharp tip and the second needle tip is a blunt tip and wherein the sharp tip is sharper than the blunt tip.

14. The compound needle of claim 13, wherein the sharp tip is retractable through the blunt tip into the hollow core of the outer shaft.

15. The compound needle of claim 9, wherein the outer shaft includes additional features that improve the echogenicity of the compound needle.

16. The compound needle of claim 15, wherein the additional features comprise indentations on the outer shaft.

17. The compound needle of claim 15, wherein the additional features comprise protruding bumps of metallic deposits on the outer shaft.

18. The compound needle of claim 9, wherein the outer shaft has an electrically insulating layer formed on an exterior surface of the outer shaft.

19. A compound needle system comprising:
a compound needle, the compound needle comprising:
an inner shaft having a first needle tip;
an outer shaft having a second needle tip;
wherein one of the first and second needle tips is sharper than the other of the and second needle tips;
wherein the outer shaft has a hollow core and wherein the inner shaft is disposed within the hollow core; and
wherein the first needle tip of the inner shaft is deployable through the second needle tip of the outer shaft; and
a visual guidance instrument wherein the visual guidance instrument is used in combination with the compound needle to treat or locate a target, the
visual guidance instrument comprising:
an external monitor device; and
a handheld probe in communication with the external monitor device.

20. The compound needle system of claim 19 wherein the visual guidance instrument is an ultrasound device and wherein the handheld probe emits into the target ultrasonic waves that reflect off the compound needle.

21. The compound needle system of claim 19 further comprising a nerve stimulation instrument wherein the nerve stimulation instrument is used in combination with the compound needle to stimulate a target, the nerve stimulation instrument comprising:
a stimulation control device;
a grounding pad; and
wherein the stimulation control device is in electrical communication with the first needle tip of the inner shaft.

22. The compound needle system of claim 21, further comprising an insulating layer wherein the insulating layer is evenly placed on the outer needle shaft.

23. A method for treating a target using a compound needle system, the method comprising:
inserting a compound needle into the skin of a patient wherein the compound needle comprises:
an inner shaft having a first needle tip;
an outer shaft having a second needle tip;
wherein one of the first and second needle tips is sharper than the other of the first and second needle tips;
wherein the outer shaft has a hollow core and wherein the inner shaft is disposed within the hollow core; and
wherein the first needle tip of the inner shaft is deployable through the second needle of the outer shaft;
advancing the compound needle to a first position;
using a visual guidance instrument to visualize the first position wherein the
visual guidance instrument comprises:
an external monitor device;
a handheld probe; and
wherein the handheld probe is in communication with the external monitor device.
deploying the first needle tip of the inner shaft;
advancing the first needle tip of the inner shaft to a second position;
using the visual guidance instrument to confirm that the second position is a proper position; and
applying therapy to the target.

24. The method of claim 23, wherein the visual guidance instrument is an ultrasound device, and further comprising:
emitting into the target ultrasonic waves that reflect off the compound needle.

25. The method of claim 23, further comprising using a nerve stimulation instrument to stimulate the target.
